(19) United States
(12) Patent Application Publication
(10) Pub. No.: US 2024/0198110 A1
(43) Pub. Date: Jun. 20, 2024

(54) SYSTEMS AND METHODS FOR MONITORING AND REVISING ELECTRICAL STIMULATION

(71) Applicant: Boston Scientific Neuromodulation Corporation, Valencia, CA (US)

(72) Inventor: Lisa Denise Moore, Glendale, CA (US)

(21) Appl. No.: 18/535,655

(22) Filed: Dec. 11, 2023

Related U.S. Application Data
(60) Provisional application No. 63/432,628, filed on Dec. 14, 2022.

(51) Int. Cl. A61N 1/36; G16H 10/20; G16H 40/67

(52) U.S. Cl. CPC A61N 1/36139; A61N 1/36132; G16H 10/20; G16H 40/67

(57) ABSTRACT

A method for monitoring and revising electrical stimulation over time includes obtaining or determining i) a therapeutic effects value and a side effects value for a first set of stimulation parameter values or ii) a therapeutic effect threshold value or side effect threshold value for an electrode selection of the first set of stimulation parameter values; performing clinical effects determinations for at least the first set of stimulation parameter values at points in time extending over at least one week; evaluating the clinical effects determinations to identify any difference from the therapeutic effects value, the side effects value, the therapeutic effect threshold value, or the side effect threshold value over time; and, when the difference meets or exceeds a threshold, recommending or implementing a new set of stimulation parameter values based on previous clinical effects determinations.

### CROSS-REFERENCE TO RELATED APPLICATIONS

This application claims the benefit under 35 U.S.C. § 119(e) of U.S. Provisional Patent Application Ser. No. 63/432,628, filed Dec. 14, 2022, which is incorporated herein by reference.

### FIELD

The present invention is directed to the area of implantable electrical stimulation systems and methods of making and using the systems. The present invention is also directed to systems and methods for monitoring and revising electrical stimulation over time.

### BACKGROUND

Implantable electrical stimulation systems have proven therapeutic in a variety of diseases and disorders. For example, deep brain stimulation can be used to treat a variety of diseases and disorders, such as Parkinson's disease, essential tremor, or the like. Spinal cord stimulation systems have been used as a therapeutic modality for the treatment of chronic pain syndromes. Peripheral nerve stimulation has been used to treat chronic pain syndrome and incontinence, with a number of other applications under investigation.

Stimulators have been developed to provide therapy for a variety of treatments. A stimulator can include a control module (with a pulse generator) and one or more stimulator electrodes. The one or more stimulator electrodes can be disposed along one or more leads, or along the control module, or both. The stimulator electrodes are in contact with or near the neural or brain tissue, nerves, muscles, or other tissue to be stimulated. The pulse generator in the control module generates electrical pulses that are delivered by the electrodes to body tissue.

### BRIEF SUMMARY

One aspect is a method for monitoring and revising electrical stimulation over time. The method includes obtaining or determining i) a therapeutic effects value and a side effects value for a first set of stimulation parameter values or ii) a therapeutic effect threshold value or side effect threshold value for an electrode selection of the first set of stimulation parameter values; performing clinical effects determinations for at least the first set of stimulation parameter values at a plurality of points in time extending over at least one week; evaluating, by a processor, the clinical effects determinations to identify any difference from the therapeutic effects value, the side effects value, the therapeutic effect threshold value, or the side effect threshold value over time; and, when the difference from the therapeutic effects value, the side effects value, the therapeutic effect threshold value, or the side effect threshold value meets or exceeds a threshold, recommending or implementing, by the processor and without human intervention, a new set of stimulation parameter values based on previous clinical effects determinations.

Another aspect is an electrical stimulation system that includes a lead comprising a plurality of electrodes disposed along a distal portion of the lead; and at least one processor configured for performing actions including obtaining or determining i) a therapeutic effects value and a side effects value for a first set of stimulation parameter values or ii) a therapeutic effect threshold value or side effect threshold value for an electrode selection of the first set of stimulation parameter values; performing clinical effects determinations for at least the first set of stimulation parameter values at a plurality of points in time extending over at least one week; evaluating, by a processor, the clinical effects determinations to identify any difference from the therapeutic effects value, the side effects value, the therapeutic effect threshold value, or the side effect threshold value over time; and, when the difference from the therapeutic effects value, the side effects value, the therapeutic effect threshold value, or the side effect threshold value meets or exceeds a threshold, recommending or implementing, by the processor and without human intervention, a new set of stimulation parameter values based on previous clinical effects determinations.

Yet another aspect is a non-transitory computer-readable medium having processor-executable instructions for monitoring stimulation drift, the processor-executable instructions when installed onto a device enable the device to perform actions, the actions including obtaining or determining i) a therapeutic effects value and a side effects value for a first set of stimulation parameter values or ii) a therapeutic effect threshold value or side effect threshold value for an electrode selection of the first set of stimulation parameter values; performing clinical effects determinations for at least the first set of stimulation parameter values at a plurality of points in time extending over at least one week; evaluating, by a processor, the clinical effects determinations to identify any difference from the therapeutic effects value, the side effects value, the therapeutic effect threshold value, or the side effect threshold value over time; and, when the difference from the therapeutic effects value, the side effects value, the therapeutic effect threshold value, or the side effect threshold value meets or exceeds a threshold, recommending or implementing, by the processor and without human intervention, a new set of stimulation parameter values based on previous clinical effects determinations.

In at least some aspects, the side effects value is indicative of no side effects or side effects below a threshold level. In at least some aspects, the threshold includes an increase in side effects above a threshold level or by a threshold amount. In at least some aspects, the threshold includes identifying, or achieving a threshold level of, a new side effect. In at least some aspects, the threshold includes a decrease in therapeutic effect below a threshold level or by a threshold amount.

A further aspect is a method for monitoring and revising electrical stimulation over time. The method includes obtaining or determining a first therapeutic effects value or first therapeutic effect threshold value for a first set of stimulation parameter values; performing a determination of a subsequent therapeutic effects value or a subsequent therapeutic threshold value for at least the first set of stimulation parameter values at a plurality of points in time extending over at least one week; evaluating, by a processor, the subsequent therapeutic effects values or subsequent therapeutic threshold values to identify a difference from the first therapeutic effects value or first therapeutic threshold value, respectively; and, when the difference from the first therapeutic effects value or first therapeutic threshold value exceeds a threshold, recommending or implementing, by the processor and without human intervention, a new set of stimulation parameter values based on previous clinical effects determinations.

Another aspect is an electrical stimulation system that includes a lead comprising a plurality of electrodes disposed along a distal portion of the lead; and at least one processor configured for performing actions including obtaining or determining a first therapeutic effects value or first therapeutic effect threshold value for a first set of stimulation parameter values; performing a determination of a subsequent therapeutic effects value or a subsequent therapeutic threshold value for at least the first set of stimulation parameter values at a plurality of points in time extending over at least one week; evaluating, by a processor, the subsequent therapeutic effects values or subsequent therapeutic threshold values to identify a difference from the first therapeutic effects value or first therapeutic threshold value, respectively; and, when the difference from the first therapeutic effects value or first therapeutic threshold value exceeds a threshold, recommending or implementing, by the processor and without human intervention, a new set of stimulation parameter values based on previous clinical effects determinations.

Yet another aspect is a non-transitory computer-readable medium having processor-executable instructions for monitoring stimulation drift, the processor-executable instructions when installed onto a device enable the device to perform actions, the actions including obtaining or determining a first therapeutic effects value or first therapeutic effect threshold value for a first set of stimulation parameter values; performing a determination of a subsequent therapeutic effects value or a subsequent therapeutic threshold value for at least the first set of stimulation parameter values at a plurality of points in time extending over at least one week; evaluating, by a processor, the subsequent therapeutic effects values or subsequent therapeutic threshold values to identify a difference from the first therapeutic effects value or first therapeutic threshold value, respectively; and, when the difference from the first therapeutic effects value or first therapeutic threshold value exceeds a threshold, recommending or implementing, by the processor and without human intervention, a new set of stimulation parameter values based on previous clinical effects determinations.

In at least some aspects, the threshold includes a decrease in therapeutic effect below a threshold level or by a threshold amount.

A further aspect is a method for monitoring and revising electrical stimulation over time. The method includes stimulating a patient using a first set of stimulation parameter values; performing a determination of side effects for the stimulation at a plurality of points in time extending over at least one week; and, when the determination exceeds a side effects threshold or identifies a new side effect, recommending or implementing, by the processor and without human intervention, a new set of stimulation parameter values based on previous clinical effects determinations.

Another aspect is an electrical stimulation system that includes a lead comprising a plurality of electrodes disposed along a distal portion of the lead; and at least one processor configured for performing actions including stimulating a patient using a first set of stimulation parameter values; performing a determination of side effects for the stimulation at a plurality of points in time extending over at least one week; and, when the determination exceeds a side effects threshold or identifies a new side effect, recommending or implementing, by the processor and without human intervention, a new set of stimulation parameter values based on previous clinical effects determinations.

Yet another aspect is a non-transitory computer-readable medium having processor-executable instructions for monitoring stimulation drift, the processor-executable instructions when installed onto a device enable the device to perform actions, the actions including stimulating a patient using a first set of stimulation parameter values; performing a determination of side effects for the stimulation at a plurality of points in time extending over at least one week; and, when the determination exceeds a side effects threshold or identifies a new side effect, recommending or implementing, by the processor and without human intervention, a new set of stimulation parameter values based on previous clinical effects determinations.

In any of the aspects described above, the method or actions can further include the method or actions can further include performing additional clinical effects determinations for a plurality of additional sets of stimulation parameter values. In any of the aspects described above, the method or actions can further include generating a clinical effects map using one or more of the clinical effects determinations and one or more of the additional clinical effects determinations. In at least some aspects, generating the clinical effects map including weighting at least one of the clinical effects determinations based on an age of the at least one of the clinical effects determinations.

In any of the aspects described above, the performing includes obtaining sensor data to assist in determining at least one of the clinical effects determinations. In any of the aspects described above, the performing includes obtaining patient feedback or patient questionnaire to assist in determining at least one of the clinical effects determinations.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is a schematic overview of one embodiment of components of a stimulation system, including an electronic subassembly disposed within a control module;

FIG. 4 is a schematic illustration of one embodiment of a three-dimensional clinical effects map;

FIG. 5 is a flowchart of one embodiment of a method for monitoring and revising electrical stimulation over time;

FIG. 6 is a flowchart of another embodiment of a method for monitoring and revising electrical stimulation over time; and

FIG. 7 is flowchart of a further embodiment of a method for monitoring and revising electrical stimulation over time.

### DETAILED DESCRIPTION

The present invention is directed to the area of implantable electrical stimulation systems and methods of making and using the systems. The present invention is also directed to systems and methods for monitoring and revising electrical stimulation over time.

Suitable implantable electrical stimulation systems include, but are not limited to, a least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Application Publications Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated herein by reference. In the discussion below, a percutaneous lead will be exemplified, but it will be understood that the methods and systems described herein are also applicable to paddle leads and other leads.

A percutaneous lead for electrical stimulation (for example, deep brain, spinal cord, or peripheral nerve stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes.

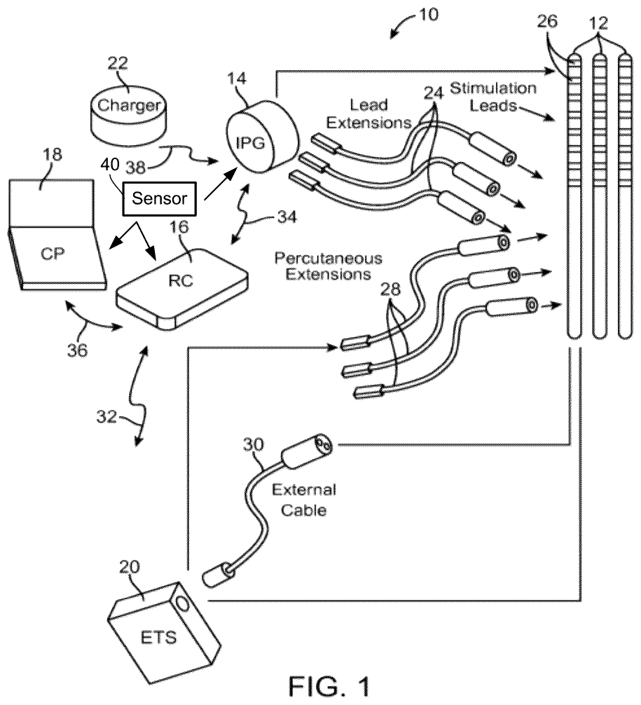

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameter values. The implantable pulse generator can be implanted into a patient's body, for example, below the patient's clavicle area or within the patient's buttocks or abdominal cavity or at any other suitable site. The implantable pulse generator can have multiple stimulation channels which may be independently programmable to control the magnitude of the current stimulus from each channel. In some embodiments, the implantable pulse generator can have any suitable number of stimulation channels including, but not limited to, 4, 6, 8, 12, 16, 32, or more stimulation channels. The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameter values. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameter values to actively control the characteristics of the electrical stimulation energy output by the IPG 14. The CP 18 allows a user, such as a clinician, the ability to program stimulation parameter values for the IPG 14 and ETS 20 in the operating room and in follow-up sessions. Alternately, or additionally, stimulation parameter values can be programed via wireless communications (e.g., Bluetooth) between the RC 16 (or external device such as a hand-held electronic device) and the IPG 14. In at least some embodiments, the RC 16 can be a mobile phone, tablet, desktop computer, or the like.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameter values provided by the CP 18 are also used to program the RC 16, so that the stimulation parameter values can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is incorporated herein by reference in its entirety. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated herein by reference in their entireties.

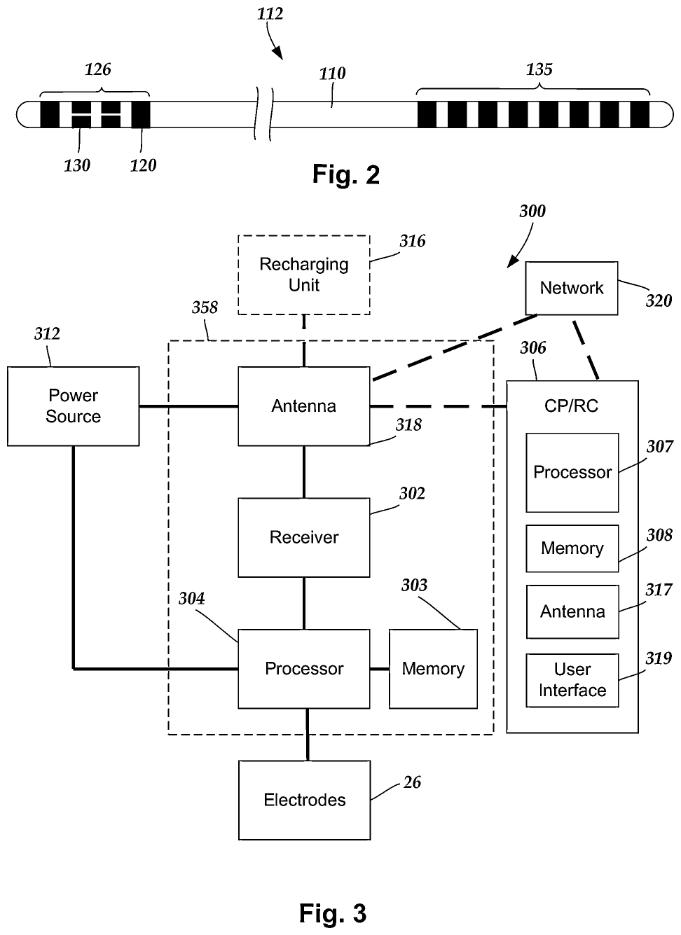

FIG. 2 illustrates one embodiment of a lead 112 with electrodes 126 disposed at least partially about a circumference of the lead 112 along a distal end portion of the lead 112 and terminals 135 disposed along a proximal end portion of the lead 112. The lead 112 can be implanted near or within the desired portion of the body to be stimulated such as, for example, the brain, spinal cord, or other body organs or tissues.

Electrodes may be disposed on the circumference of the lead 112 to stimulate the target neurons, neural tissue, brain tissue, or other tissue. Stimulation electrodes may be ring shaped so that current projects from each electrode radially from the position of the electrode along a length of the lead 112. In the embodiment of FIG. 2, two of the electrodes 126 are ring electrodes 120. Ring electrodes typically do not enable stimulus current to be directed from only a limited angular range around a lead. Segmented electrodes 130, however, can be used to direct stimulus current to a selected angular range around a lead. When segmented electrodes are used in conjunction with an implantable pulse generator that delivers constant current stimulus, current steering can be achieved to deliver the stimulus more precisely to a position around an axis of a lead (i.e., radial positioning around the axis of a lead). To achieve current steering, segmented electrodes can be utilized in addition to, or as an alternative to, ring electrodes.

The lead 112 includes a lead body 110, terminals 135, at least one ring electrode 120, and at least one set of segmented electrodes 130 (or any other combination of electrodes). The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 112 may be in contact with body tissue for extended periods of time. In at least some embodiments, the lead 112 has a cross-sectional diameter of no more than 1.5 mm and may be in the range of 0.5 to 1.5 mm. In at least some embodiments, the lead 112 has a length of at least 10 cm and the length of the lead 112 may be in the range of 10 to 70 cm.

The electrodes 126 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes 126 are made of a material that is biocompatible and does not substantially corrode under expected operating conditions in the operating environment for the expected duration of use.

Each of the electrodes 126 can either be used or unused (OFF). When an electrode is used, the electrode can be used as an anode or cathode and carry anodic or cathodic current. In some instances, an electrode might be an anode for a period of time and a cathode for a period of time.

Segmented electrodes may provide for superior current steering than ring electrodes because target structures are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Pat. Nos. 8,473,061; 8,571,665; 8,792,993; 9,248,272; 9,775,988; and 10,286,205; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; and 2015/0051681, all of which are incorporated herein by reference.

One or more percutaneous leads, such as lead 12 in FIG. 1 or lead 112 in FIG. 2, can be implanted for stimulation. In at least some embodiments, multiple percutaneous leads can be implanted and spaced apart from each other. Such an arrangement can be useful for, for example, spinal cord stimulation to stimulation two regions of the spinal cord or for deep brain stimulation to stimulate opposite hemispheres of the brain. For example, one or more percutaneous leads can be implanted on each lateral side of the spinal cord and arranged over or near the dorsal columns or dorsal horns. Optionally, a medial percutaneous lead may also be implanted.

FIG. 3 is a schematic overview of one embodiment of components of an electrical stimulation system 300 including an electronic subassembly 310 disposed within an IPG 14 (FIG. 1). It will be understood that the electrical stimulation system can include more, fewer, or different components and can have a variety of different configurations including those configurations disclosed in the stimulator references cited herein.

The IPG 14 (FIG. 1) can include, for example, a power source 312, antenna 318, receiver 302, processor 304, and memory 303. Some of the components (for example, power source 312, antenna 318, receiver 302, processor 304, and memory 303) of the electrical stimulation system can be positioned on one or more circuit boards or similar carriers within a sealed housing of the IPG 14 (FIG. 1), if desired. Unless indicated otherwise, the term “processor” refers to both embodiments with a single processor and embodiments with multiple processors.

An external device, such as a CP or RC 306, can include a processor 307, memory 308, an antenna 317, and a user interface 319. The user interface 319 can include, but is not limited to, a display screen on which a digital user interface can be displayed and any suitable user input device, such as a keyboard, touchscreen, mouse, track ball, or the like or any combination thereof.

Any power source 312 can be used including, for example, a battery such as a primary cell battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

If the power source 312 is rechargeable battery, the battery may be recharged using the antenna 318, if desired. Power can be provided to the battery for recharging by inductively coupling the battery through the antenna to an optional recharging unit 316 external to the user. Examples of such arrangements can be found in the references identified above.

In one embodiment, electrical current is emitted by the electrodes 26 on the lead body to stimulate nerve fibers, muscle fibers, or other body tissues near the electrical stimulation system. A processor 304 is generally included to control the timing and electrical characteristics of the electrical stimulation system. For example, the processor 304 can, if desired, control one or more of the timing, frequency, amplitude, width, and waveform of the pulses. In addition, the processor 304 can select which electrodes can be used to provide stimulation, if desired. In some embodiments, the processor 304 may select which electrode(s) are cathodes and which electrode(s) are anodes. In some embodiments, the processor 304 may be used to identify which electrodes provide the most useful stimulation of the desired tissue. Instructions for the processor 304 can be stored on the memory 303. Instructions for the processor 307 can be stored on the memory 308.

Any processor 304 can be used for the IPG and can be as simple as an electronic device that, for example, produces pulses at a regular interval or the processor can be capable of receiving and interpreting instructions from the CP/RC 306 (such as CP 18 or RC 16 of FIG. 1) that, for example, allows modification of pulse characteristics. In the illustrated embodiment, the processor 304 is coupled to a receiver 302 which, in turn, is coupled to the antenna 318. This allows the processor 304 to receive instructions from an external source to, for example, direct the pulse characteristics and the selection of electrodes, if desired. Any suitable processor 307 can be used for the CP/RC 306.

Any suitable memory 303, 308 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In one embodiment, the antenna 318 is capable of receiving signals (e.g., RF signals) from an antenna 317 of a CP/RC 306 (see, CP 18 or RC 16 of FIG. 1) which is programmed or otherwise operated by a user. The signals sent to the processor 304 via the antenna 318 and receiver 302 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 300 to cease operation, to start operation, to start signal acquisition, or to stop signal acquisition. In other embodiments, the stimulation system does not include an antenna 318 or receiver 302 and the processor 304 operates as programmed.

Optionally, the electrical stimulation system 300 may include a transmitter (not shown) coupled to the processor 304 and the antenna 318 for transmitting signals back to the CP/RC 306 or another unit capable of receiving the signals. For example, the electrical stimulation system 300 may transmit signals indicating whether the electrical stimulation system 300 is operating properly or not or the level of charge remaining in the battery. The processor 304 may also be capable of transmitting information about the pulse characteristics so that a user or clinician can determine or verify the characteristics.

Transmission of signals can occur using any suitable method, technique, or platform including, but not limited to, inductive transmission, radiofrequency transmission, Bluetooth™, Wi-Fi, cellular transmission, near field transmission, infrared transmission, or the like or any combination thereof. In addition, the IPG 14 can be wirelessly coupled to the RC 16 or CP 18 using any suitable arrangement include direct transmission or transmission through a network, such as a local area network, wide area network, the Internet, or the like or any combination thereof. The CP 18 or RC 16 may also be capable of coupling to, and sending data or other information to, a network 320, such as a local area network, wide area network, the Internet, or the like or any combination thereof.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (for example, RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

Deep Brain Stimulation (DBS) has been shown to alleviate the symptoms of a variety of diseases or disorders, such as, for example, Parkinson's Disease, essential tremor, Alzheimer's disease, or the like. As an example, Parkinson's Disease is a progressive neurodegenerative disease with no lasting cure. As a result, as the disease progresses stimulation will likely be altered to adapt to the changing conditions within the brain.

In addition, the effectiveness of the primary stimulation position may change over time due to stimulation drift. Stimulation drift may occur for a variety of reasons, such as, for example, migration of the stimulation lead over time or migration of the stimulation target if the stimulated tissue changes physiologically or becomes desensitized.

In at least some embodiments, it is desirable to monitor these changing conditions of the disease or disorder, stimulation drift, or any other effects that may alter the effectiveness of the stimulation. In at least some embodiments, it is desirable to alter the stimulation parameter values to change the primary stimulation position along the lead or to alter stimulation to improve effectiveness or avoid/reduce new or worsening side effects.

Systems and methods are described herein that identify or recognize changes in clinical effects over time and provide recommendations, in view of the changes, for stimulation programs and stimulation parameter values. When an electrical stimulation system is programmed, the programmer (for example, a clinician, patient, or other individual) selects stimulation parameter values, including the selection of electrode(s) and corresponding amplitude(s), that provide a stimulation benefit. Other stimulation parameters include, but are not limited, to pulse width, pulse frequency, stimulation duration, pulse waveform, electrode polarity, or the like. In at least some embodiments, different sets of stimulation parameter values are tested to determine which set of stimulation parameter values provides a desired stimulation benefit (which may also include the absence, or lower severity, of side effects.) This set of programmed stimulation parameter values can be thought of as providing stimulation from a primary stimulation position along the lead.

In at least some embodiments, the selection of one or more electrodes for stimulation, as well as the selection of the stimulation amplitude for each electrode, determines the stimulation position along the lead. In at least some embodiments, the stimulation position corresponds to a virtual electrode from which the electrical stimulation appears to arise for the particular electrode selection and stimulation amplitude(s). In at least some embodiments, the stimulation position can correspond to a composite of the selected electrode(s).

The selection of parameters for stimulation, such as deep brain electrical stimulation, can include the determination of clinical effects for different sets of stimulation parameter values. The determination of clinical effects can include the variation, selection, alteration, or modification of one or more parameters such as, for example, electrode selection, stimulation amplitude, fractionalization (i.e., the distribution of the stimulation amplitude among the electrodes), electrode polarity (e.g., cathode or anode), stimulation duration, pulse frequency, pulse width, waveform, or the like or any combination thereof. The clinical effects can be therapeutic effects, therapeutic responses, side effects, testing results (e.g., results of one or more testing protocols), or any combination thereof. It will be understood that the term “clinical effects” and terms “responses” and “clinical responses” are interchangeable herein.

A clinical effects map that displays one or more therapeutic effects, therapeutic responses, side effects, or test results (or any combination thereof) for each of one or more stimulation instances (or estimated clinical effects for stimulation instances) can guide stimulation programming. One example of a two-dimensional clinical effects map is described in U.S. Patent Application Publication No. 2014/0277284, incorporated herein by reference in its entirety. In this particular example, the x axis of the clinical effects map corresponds to stimulation amplitude and the y axis corresponds to the position (or a composite position) of the stimulating electrode(s) along the lead. In this particular example, the y axis provides spatial information regarding the stimulation, but the x axis does not. One example of a three-dimensional clinical effects map is described in U.S. Pat. No. 10,071,249, incorporated herein by reference in its entirety. The x and z axes correspond to the electrode selection and the y axis corresponds to the amplitude. Two of the axes provide spatial information but this spatial information corresponds to the position (or a composite position) of one or more stimulating electrodes on the lead. U.S. Provisional Patent Application Ser. Nos. 63/288,153 and 63/425,149, both of which are incorporated herein by reference in its entirety, illustrates other embodiments of a clinical effects map.

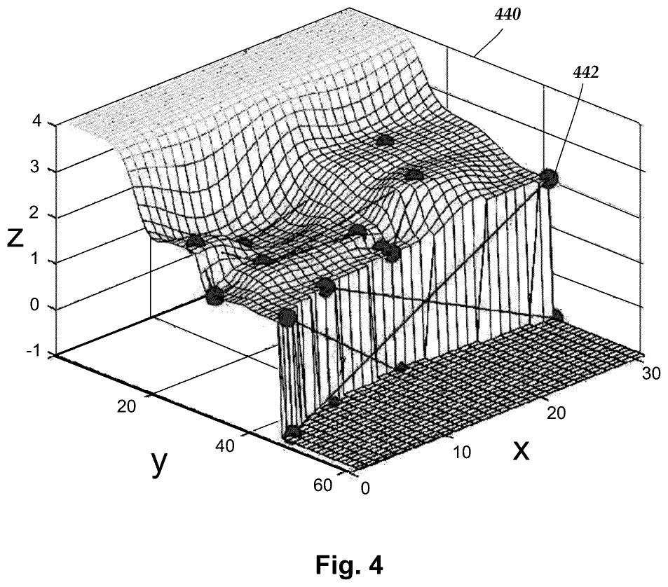

FIG. 4 illustrates another three-dimensional clinical effects map 440 in which the x direction represents position along the lead (from the distal-most electrode at 0 to the proximal-most electrode at 4) and indicates the electrode selection, the y axis represents stimulation amplitude (in tenths of a milliamp), and the z axis represents clinical effects ranging from “4” as the worst (or no) therapeutic effect to “0” as the best therapeutic effect and “−1” indicates the presence of a side effect (or a side effect above a threshold level). The spheres 442 in FIG. 4 represent sets of stimulation parameter values that were tested.

A clinical effects map is typically generated from multiple stimulation instances. Each stimulation instance is defined by one or more sets of stimulation parameter values (for example, electrode selection, stimulation amplitude, pulse width, pulse frequency, or the like) that are used for the stimulation. In at least some embodiments, the stimulation parameter values for the stimulation instances can be manually programmed or the stimulation instances can be a set of stimulations performed using an automated programming sequence or any combination thereof. In at least some embodiments, an automated programming sequence may also utilize the clinical effects from preceding stimulation instances to inform or select the next or succeeding stimulation instances. U.S. Pat. No. 10,603,498, incorporated herein by reference in its entirety, describes examples of such automated programming.

For each stimulation instance, a measure or score for at least one clinical effect is obtained or determined and then recorded. The observation, determination, or input of the clinical effect(s) may be performed by the user, the patient, or any other suitable person or the clinical effect(s) can be observed or determined by a processor of the system or a sensor or other device.

Examples of measurements or scores for clinical effects include, but are not limited to, manually assessed clinical scores (for example, scores determined using observations, questionnaires, patient feedback, or the like), sensor-derived scores or values, electrophysiological signals, or the like or any combination thereof. For example, a user may input a quantitative or qualitative score based on visual observation of the patient, a sensor (either internal or external), or data (for example, an EEG or ECG or the like); verbal feedback from the patient; an evoked compound action potential (ECAP) or an evoked resonant neural activity (ERNA); or the like. Manually assessed clinical scores or observations and automated assessments can include the patient performing motor or other tasks in front of clinician or camera with clinician or automated scoring or assessment of the patient's speech or performance of cognitive tasks.

As another example, at least one internal or external sensor (for example, a haptic sensor, accelerometer, gyroscope, EEG, EMG, camera, or the like) may be used to observe or determine one or more clinical effects and may provide a quantitative or qualitative measurement, score, or other value (either directly to the processor or through a programmer, a user, the patient, or another person) that represents one of the clinical effects. A quantitative or qualitative value can indicate, for example, at least one characteristic of a symptom (for example, tremor), a therapeutic effect or side effect (for example, change in the patient's balance), electrical activity, or the like. The clinical effect may be indicative of a therapeutic effect or a side effect or both. Moreover, in at least some embodiments, more than one clinical effect can be observed, determined, or input for each stimulation instance.

The clinical effects map 440 of FIG. 4 illustrates the measurement of clinical effects at multiple points followed by extrapolation of other points within the clinical effects map. Any suitable extrapolation method or technique can be used.

A clinical effects map can facilitate identifying one or more sets of stimulation parameter values that provide a good response and sets of stimulation parameter values that do not provide a good response (or may even provide a poor response or elicit side effects). A clinical effects map may also identify sets of stimulation parameter values that produce side effects or produce side effects at or above a threshold.

In at least some embodiments, the systems and methods utilize sets of clinical effects data (for example, repeated clinical effects determinations or clinical effects maps) obtained over time to achieve these objections. The clinical effects data can be the generation of a clinical effects map at different times or can be simply clinical effects determinations for one or more sets of stimulation parameter values repeated over time. In at least some embodiments, the clinical effects data can be repeated determinations of the clinical effects for one or more sets of parameters used for one or more stimulation programs. Such an arrangement can include monitoring the effectiveness of therapeutic stimulation programs over time.

The time period between clinical effects determinations can be any suitable time period including, but not limited to, 1, 5, 10, 15, 20, 30, or 45 minutes or 1, 2, 3, 4, 6, 12, or 18 hours, 1, 2, 3, 4, 5, 6, 7, 10, 14, 15, 21, or 28 days or 1, 2, 3, 4, 6, 8, 10, or 12 months or any other suitable period between or exceeding those recited herein. In at least some embodiments, multiple clinical effects determinations may be made over a period of, for example, 5, 10, 15, 20, 30, or 45 minutes or 1, 2, 3, 4, 6, 12, or 18 hours, 1, 2, 3, 4, 5, 6, 7, 10, 14, 15, 21, or 28 days or 1, 2, 3, 4, 6, 8, 10, 12, 15, 18, 24, 30, 36, 48, 50, or 60 months or more or any other suitable period between or exceeding those recited herein. In at least some embodiments, some or all of the clinical effects determinations are made at regular intervals. In at least some embodiments, the clinical effects determinations are automated. In at least some embodiments, some or all of the clinical effects determinations are made at irregular intervals or at the request of a clinician, patient, or other individual. The requests may be made by the same individual or different individuals. The requests may be made using the RC 16, CP 18, a clinician portal, a smartphone, a smart watch, or any other suitable device.

The clinical effects determinations can be performed using the RC 16, CP 18, sensor 40, an application on a mobile phone or wearable device (such as a smart watch), or any other suitable device. The clinical effects determinations can be initiated by the patient, clinician, or any other suitable individual.

In at least some embodiments, the systems and methods described herein can identify trends in clinical effects data. In at least some embodiments, these trends are patient specific. In at least some embodiments, similar trends may be identified for a cohort of patients have one or more common features and such cohort trends can also be used to provide recommendations for stimulation programs and stimulation parameter values for other members of the cohort or other patients.

In at least some embodiments, the systems or methods compare two or more sets of clinical effects data (for example, clinical effects maps or graphs, clinical effects determinations, or the like) recorded at different times for the same patient. In at least some embodiments, the systems or methods compare two or more sets of clinical effects data (for example, clinical effects maps or graphs, clinical effects determinations, or the like) recorded for different patients or for a cohort of patients. In at least some embodiments, such comparisons between clinical effects data from different patients can be used to predict the course of stimulation or the disease for a particular patient. In at least some embodiments, such comparisons between clinical effects data from different patients can produce recommendations for the particular patient based on experiences from other patients and the correlation of those experiences to the progression of the clinical effects data for the particular patient.

In at least some embodiments, the systems and methods analyze the sets of clinical effects data to identify elements such as, for example, changes in side effect thresholds or therapeutic effect thresholds, changes in the amount or intensity of therapeutic effects or side effects (e.g., a therapeutic effects value or a side effects value) for a particular set of stimulation parameter values, medication state, or the like or any combination thereof.

A therapeutic effects value or a side effects value is a value that reflects the intensity of the therapeutic effect or side effect for a given set of stimulation parameter values. Turning to FIG. 4, the therapeutic effects value or side effects values is the z-axis value indicated in the clinical effects map for a particular selection of electrode(s) (x-axis) and a particular stimulation amplitude (y-axis). As an example, in FIG. 4, the therapeutic effects value for an electrode selection of “0” (e.g., the distal-most electrode) and a stimulation amplitude selection of “30” (e.g., 3 mA) is “1”.

A side effect threshold value or therapeutic effect threshold value corresponds to a value for a stimulation parameter (for example, stimulation amplitude) that elicits the side effect or therapeutic effect for a particular selection of electrode(s). Turning to FIG. 4, the side effect threshold value or therapeutic effect threshold value can be, for example, the lowest stimulation amplitude (y-axis) at which a particular therapeutic effect value or side effect value (z-axis) is achieved for a particular selection of electrode(s) (x-axis). As an example, in FIG. 4, the therapeutic effect threshold value for an electrode selection of “0” (e.g., the distal-most electrode) and a therapeutic effects value of “2” is approximately “20” (e.g., approximately 2 mA) and the side effect threshold value (where the z-axis value is “−1”) is approximately “4.5” (e.g., approximately 4.5 mA).

In at least some embodiments, the side effect or therapeutic effect is elicited at a particular level (for example, at a level noticeable to the patient, uncomfortable for the patient, or providing substantial relief to the patient for a particular symptom or symptoms) in order to determine the side effect or therapeutic effect threshold. In FIG. 4, that threshold for therapeutic effects might be “3” or “2” on the z-axis and the threshold for side effects is “−1”.

In at least some embodiments, the clinical effects data related to a side effect is updated when there is a change in a side effect threshold that suggests a consistent decrease or increase in the stimulation amplitude that elicits the side effect. In at least some embodiments, the systems or methods can recommend one or more new sets of stimulation parameter values in view of the increase or decrease of the stimulation amplitude that elicits the side effect.

In at least some embodiments, a side effect region can be established with a probability assigned based on how often a side effect has been recorded at a given amplitude or lower when the side effect threshold appears to vary in amplitude is not consistently trending up or down. In at least some embodiments, the systems or methods can recommend one or more new sets of stimulation parameter values in view of variation in the side effect threshold or the assigned probability.

In at least some embodiments, the systems or methods can identify a new side effect and recommend one or more new sets of stimulation parameter values to avoid this new side effect. In at least some instances, a new side effect may not be evident in clinic or during a programming session but arise after longer stimulation or from a result of neurodegeneration that has occurred since the last clinical visit. As an example, a patient or sensor may record mood disturbances, sleep disturbance, or increased fall incidence arising from longer use of the stimulation program. The region stimulated by the current set of stimulation parameter values can be marked as inducing, or potentially inducing, a side effect. The system or method can recommend one or more new sets of stimulation parameter values that avoid, or reduce stimulation of, the identified region.

In at least some embodiments, the systems or methods can identify diminishing therapeutic effect and recommend one or more new sets of stimulation parameter values that are likely to increase the therapeutic effect. For example, the systems or methods can identify regions of the clinical effects map that produce a better therapeutic response or are projected to produce a better therapeutic response.

In at least some embodiments, when recommending one or more new sets of stimulation parameter values, the systems or methods may utilize a clinical effects map that includes current clinical effects determinations and clinical effects determinations obtained previously. In at least some embodiments, previous clinical effects determinations may be ignored when more recent clinical effects determinations are available for the same set of stimulation parameter values or, in some embodiments, similar sets of stimulation parameter values (where similarity is defined, or definable, by the system or user). In at least some embodiments, previous clinical effects determinations may be weighted based on the time the clinical effects determination was obtained so that more recent clinical effects determinations are considered more reliable. Clinical determination may be weighted based on the therapeutic effect or side effect so that selected therapeutic effect(s) or side effect(s) are more highly weighted than other.

In at least some embodiments, when recommending one or more new sets of stimulation parameter values, the systems or methods may test a number of sets of stimulation parameter values and obtain clinical effects for each set. In at least some embodiments, the clinical effects can be obtained using one or more sensors, patient feedback, patient questionnaire, or the like or any combination thereof. In at least some embodiments, the testing may be restricted to avoid sets of stimulation parameter values that are known or likely to cause side effects. In at least some embodiments, the testing may be restricted with respect to values for particular stimulation parameter values, such as stimulation amplitude. For example, the maximum value of the stimulation amplitude may be restricted to avoid causing discomfort or damage to the patient.

In at least some embodiments, the system or methods can generate a clinical effects map based on clinical effects determinations made in response to the need or request for a recommendation and, optionally, previous clinical effects determinations. In at least some embodiments, the system or methods may limit or reject previous clinical effects determinations that are older than a defined or selected time.

Examples of an automated arrangement for providing recommendations for a new set of stimulation parameter values based on the clinical effects from previous stimulations can be found in U.S. Pat. No. 10,603,498, incorporated herein by reference in its entirety.

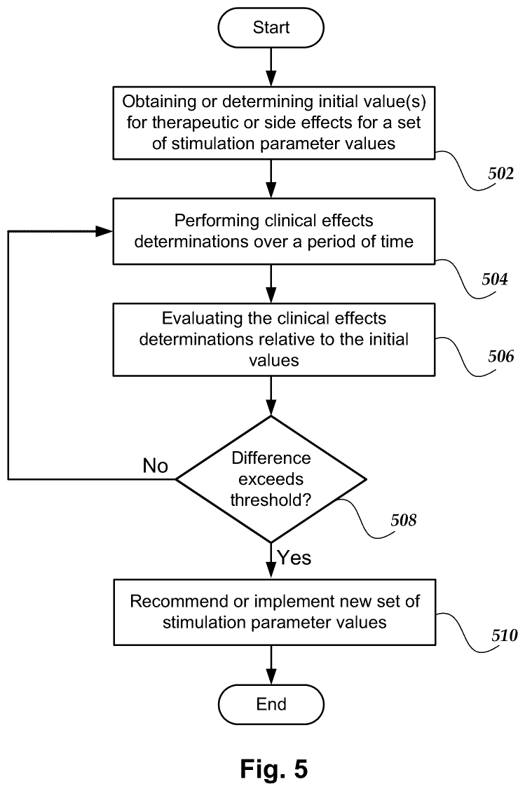

FIG. 5 is a flowchart of one embodiment of a method for monitoring and revising electrical stimulation over time. In step 502 one or more values relating to therapeutic or side effects are obtained or determined for at least one set of stimulation parameter values. For example, a therapeutic effects value and a side effects value can be obtained or determined for at least one set of stimulation parameter values. As described above, these values are a measurement or other indication of the value or amount of therapeutic or side effects elicited by stimulation using the set of stimulation parameter values.

As another example, a therapeutic effect threshold value or side effect threshold value for the electrode selection of each of at least one of stimulation parameter values is obtained or determined. As described above, these values can correspond to a lowest stimulation amplitude (or other stimulation parameter) that elicits a threshold amount of therapeutic or side effects for the particular electrode selection.

The values for therapeutic or side effects can be obtained or determined using any suitable method including, but not limited to, from one or sensors (e.g., implanted or wearable sensors or any combination thereof), from the patient or clinician (or any other suitable individual), from a clinical effects map or clinical effects data, or the like or any combination thereof. The values can be obtained for a single set of stimulation parameter values or multiple sets of stimulation parameter values.

In step 504, clinical effects determinations are performed over a period of time (for example, at least 1, 2, 3, 4, 5, 7, or more days, 1, 2, 3, 4, or more weeks, 1, 2, 3, 4, 6, 8, 10, or more months, 1, 2, 3, 4, 5, or more years, or any other suitable period of time between or beyond those listed herein) as the patient is stimulated using one or more sets of stimulation parameter values. In at least some embodiments, the clinical effects determinations can include determining any of the values described above with respect to step 502 using any of the methods described above with respect to step 502.

In step 506, the clinical effects determinations are evaluated to identify any difference from the therapeutic effects value, the side effects value, the therapeutic effect threshold value, or the side effect threshold value over time. In at least some embodiments, this evaluation is performed by a processor of the electrical stimulation system, such as a processor that is part of the RC 16, CP 18, ETS 20, IPG 14, or any other suitable device or any combination thereof. The evaluation can include comparing the values from step 502 and the values obtained during step 504. In at least some embodiments, the comparison can include determining a difference between the values from step 502 and the values obtained during step 504. Any other suitable evaluation method can be used.

In step 508, a query is made whether the values obtained during step 504 differ from the values from step 502 by at least a threshold amount. If no, then the method repeats steps 504 to 508 until an end condition is met.

If yes, then in step 510 a new set of stimulation parameter values based on previous clinical effects determinations is recommended or implemented. In at least some embodiments, the new set of stimulation parameter values is recommended or implemented by a processor, such as a processor that is part of the RC 16, CP 18, ETS 20, IPG 14, or any other suitable device or any combination thereof. In at least some embodiments, the recommendation or implementation is performed by the processor without human intervention. In at least some embodiments, a recommendation is presented by the processor, with or without human intervention, to a patient, clinician, or other individual using, for example, the RC 16, CP 18, ETS 20, IPG 14, or any other suitable device or any combination thereof. The patient, clinician, or other individual can be queried to confirm that the stimulation should proceed using the new set of stimulation parameters values. When confirmed, the system (for example, the IPG 14) can initiate stimulation using the new set of stimulation parameters values. Alternatively, the patient, clinician, or other individual can initiate stimulation using the new set of stimulation parameters values. In at least some embodiments, the method may then repeat starting at step 502.

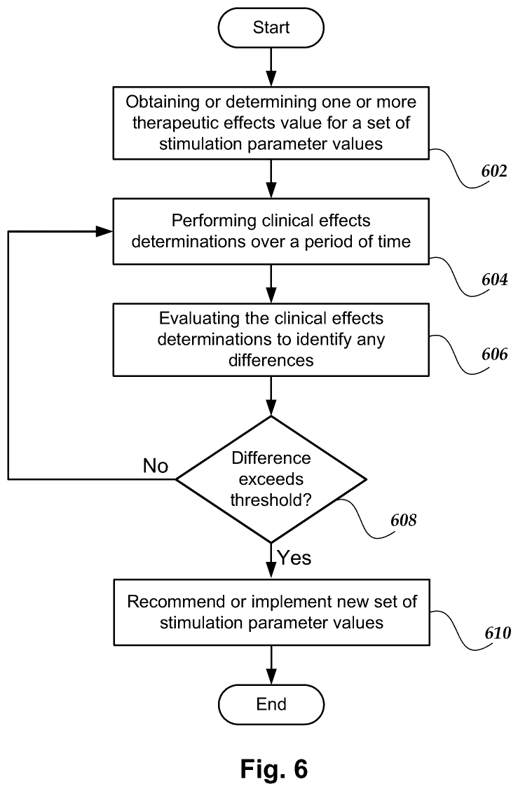

FIG. 6 is a flowchart of another embodiment of a method for monitoring and revising electrical stimulation over time. In step 602 at least one value relating to therapeutic effects is obtained or determined for at least one set of stimulation parameter values. For example, a therapeutic effects value can be obtained or determined for at least one set of stimulation parameter values. As described above, this value is a measurement or other indication of the value or amount of therapeutic effects elicited by stimulation using the set of stimulation parameter values.

As another example, a therapeutic effect threshold value for the electrode selection of each of at least one of stimulation parameter values is obtained or determined. As described above, this value can correspond to a lowest stimulation amplitude (or other stimulation parameter) that elicits a threshold amount of therapeutic effects for the particular electrode selection.

The value(s) for therapeutic effects can be obtained or determined using any suitable method including, but not limited to, from one or sensors (e.g., implanted or wearable sensors or any combination thereof), from the patient or clinician (or any other suitable individual), from a clinical effects map or clinical effects data, or the like or any combination thereof. The value(s) can be obtained for a single set of stimulation parameter values or multiple sets of stimulation parameter values.

In step 604, clinical effects determinations are performed over a period of time (for example, at least 1, 2, 3, 4, 5, 7, or more days, 1, 2, 3, 4, or more weeks, 1, 2, 3, 4, 6, 8, 10, or more months, 1, 2, 3, 4, 5, or more years, or any other suitable period of time between or beyond those listed herein) as the patient is stimulated using one or more sets of stimulation parameter values. In at least some embodiments, the clinical effects determinations can include determining any of the values described above with respect to step 602 using any of the methods described above with respect to step 602.

In step 606, the clinical effects determinations are evaluated to identify any difference from the therapeutic effects value, the side effects value, the therapeutic effect threshold value, or the side effect threshold value over time. In at least some embodiments, this evaluation is performed by a processor of the electrical stimulation system, such as a processor that is part of the RC 16, CP 18, ETS 20, IPG 14, or any other suitable device or any combination thereof. The evaluation can include comparing the values from step 602 and the values obtained during step 604. In at least some embodiments, the comparison can include determining a difference between the values from step 602 and the values obtained during step 604. Any other suitable evaluation method can be used.

In step 608, a query is made whether the values obtained during step 604 differ from the values from step 602 by at least a threshold amount. If no, then the method repeats steps 604 to 608 until an end condition is met.

If yes, then in step 610 a new set of stimulation parameter values based on previous clinical effects determinations is recommended or implemented. In at least some embodiments, the new set of stimulation parameter values is recommended or implemented by a processor, such as a processor that is part of the RC 16, CP 18, ETS 20, IPG 14, or any other suitable device or any combination thereof. In at least some embodiments, the recommendation or implementation is performed by the processor without human intervention. In at least some embodiments, a recommendation is presented by the processor, with or without human intervention, to a patient, clinician, or other individual using, for example, the RC 16, CP 18, ETS 20, IPG 14, or any other suitable device or any combination thereof. The patient, clinician, or other individual can be queried to confirm that the stimulation should proceed using the new set of stimulation parameters values. When confirmed, the system (for example, the IPG 14) can initiate stimulation using the new set of stimulation parameters values. Alternatively, the patient, clinician, or other individual can initiate stimulation using the new set of stimulation parameters values. In at least some embodiments, the method may then repeat starting at step 602.

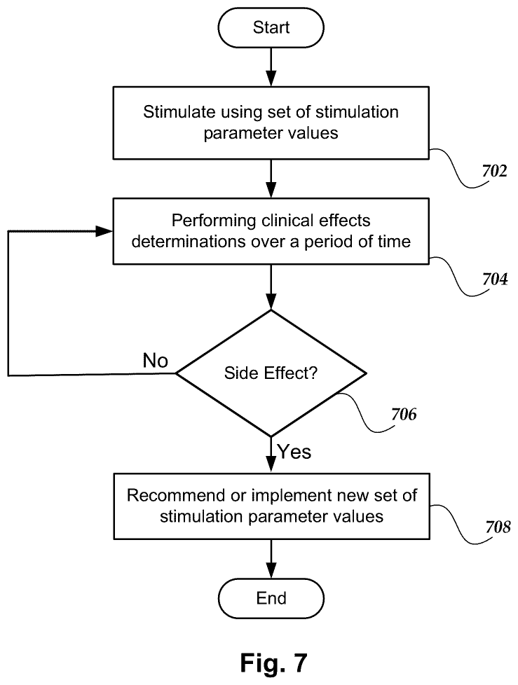

FIG. 7 is a flowchart of a further embodiment of a method for monitoring and revising electrical stimulation over time. In step 702, the patient is stimulated using at least one set of stimulation parameter values.

In step 704, clinical effects determinations are performed over a period of time (for example, at least 1, 2, 3, 4, 5, 7, or more days, 1, 2, 3, 4, or more weeks, 1, 2, 3, 4, 6, 8, 10, or more months, 1, 2, 3, 4, 5, or more years, or any other suitable period of time between or beyond those listed herein) as the patient is stimulated using one or more sets of stimulation parameter values. In at least some embodiments, the clinical effects determinations can include determining any of the values described above with respect to step 502 or 602 using any of the methods described above with respect to step 502 or 602.

In step 706, a query is made regarding a side effect. The query can include one or more of the following: whether a side effect threshold has been met, whether a new side effect has been detected, or whether a side effect has worsened. Any other suitable query can be used instead or, or in conjunction with, one or more of these queries to identify changes in the side effects experienced by the patient. If the answer to the query is no, then the method repeats steps 704 and 706 until an end condition is met.

If yes, then in step 708 a new set of stimulation parameter values based on previous clinical effects determinations is recommended or implemented. In at least some embodiments, the new set of stimulation parameter values is recommended or implemented by a processor, such as a processor that is part of the RC 16, CP 18, ETS 20, IPG 14, or any other suitable device or any combination thereof. In at least some embodiments, the recommendation or implementation is performed by the processor without human intervention. In at least some embodiments, a recommendation is presented by the processor, with or without human intervention, to a patient, clinician, or other individual using, for example, the RC 16, CP 18, ETS 20, IPG 14, or any other suitable device or any combination thereof. The patient, clinician, or other individual can be queried to confirm that the stimulation should proceed using the new set of stimulation parameters values. Alternatively, the patient, clinician, or other individual can initiate stimulation using the new set of stimulation parameters values. When confirmed, the system (for example, the IPG 14) can initiate stimulation using the new set of stimulation parameters values. In at least some embodiments, the method may then repeat starting at step 702.

It will be understood that each block of the flowcharts, and combinations of blocks in the flowcharts and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A method for monitoring and revising electrical stimulation over time, the method comprising:
obtaining or determining i) a therapeutic effects value and a side effects value for a first set of stimulation parameter values or ii) a therapeutic effect threshold value or side effect threshold value for an electrode selection of the first set of stimulation parameter values;
performing clinical effects determinations for at least the first set of stimulation parameter values at a plurality of points in time extending over at least one week;
evaluating, by a processor, the clinical effects determinations to identify any difference from the therapeutic effects value, the side effects value, the therapeutic effect threshold value, or the side effect threshold value over time; and
when the difference from the therapeutic effects value, the side effects value, the therapeutic effect threshold value, or the side effect threshold value meets or exceeds a threshold, recommending or implementing, by the processor and without human intervention, a new set of stimulation parameter values based on previous clinical effects determinations.

2. The method of claim 1, wherein the side effects value is indicative of no side effects or side effects below a threshold level.

3. The method of claim 1, wherein the threshold comprises an increase in side effects above a threshold level or by a threshold amount.

4. The method of claim 1, wherein the threshold comprises identifying, or achieving a threshold level of, a new side effect.

5. The method of claim 1, wherein the threshold comprises a decrease in therapeutic effect below a threshold level or by a threshold amount.

6. The method of claim 1, further comprising receiving a request to perform a one of the clinical effects determinations.

7. The method of claim 1, further comprising performing additional clinical effects determinations for a plurality of additional sets of stimulation parameter values.

8. The method of claim 1, further comprising generating a clinical effects map using one or more of the clinical effects determinations and one or more of the additional clinical effects determinations.

9. The method of claim 8, wherein generating the clinical effects map comprising weighting at least one of the clinical effects determinations based on an age of the at least one of the clinical effects determinations.

10. The method of claim 1, wherein the performing comprises obtaining sensor data to assist in determining at least one of the clinical effects determinations.

11. The method of claim 1, wherein the performing comprises obtaining patient feedback or patient questionnaire to assist in determining at least one of the clinical effects determinations.

12. A non-transitory computer-readable medium having processor-executable instructions for monitoring stimulation drift, the processor-executable instructions when installed onto a device enable the device to perform actions, the actions comprising the method of claim 1.

13. A method for monitoring and revising electrical stimulation over time, the method comprising:
obtaining or determining a first therapeutic effects value or first therapeutic effect threshold value for a first set of stimulation parameter values;
performing a determination of a subsequent therapeutic effects value or a subsequent therapeutic threshold value for at least the first set of stimulation parameter values at a plurality of points in time extending over at least one week;
evaluating, by a processor, the subsequent therapeutic effects values or subsequent therapeutic threshold values to identify a difference from the first therapeutic effects value or first therapeutic threshold value, respectively; and
when the difference from the first therapeutic effects value or first therapeutic threshold value exceeds a threshold, recommending or implementing, by the processor and without human intervention, a new set of stimulation parameter values based on previous clinical effects determinations.

14. The method of claim 13, wherein the threshold comprises a decrease in therapeutic effect below a threshold level or by a threshold amount.

15. The method of claim 13, further comprising generating a clinical effects map using one or more of the clinical effects determinations and one or more of the additional clinical effects determinations.

16. A non-transitory computer-readable medium having processor-executable instructions for monitoring stimulation drift, the processor-executable instructions when installed onto a device enable the device to perform actions, the actions comprising the method of claim 13.

17. A method for monitoring and revising electrical stimulation over time, the method comprising:
stimulating a patient using a first set of stimulation parameter values;
performing a determination of side effects for the stimulation at a plurality of points in time extending over at least one week; and
when the determination exceeds a side effects threshold or identifies a new side effect, recommending or implementing, by the processor and without human intervention, a new set of stimulation parameter values based on previous clinical effects determinations.

18. The method of claim 17, further comprising generating a clinical effects map using one or more of the clinical effects determinations and one or more of the additional clinical effects determinations.

19. The method of claim 17, wherein the performing comprises obtaining sensor data to assist in determining at least one of the clinical effects determinations.

20. The method of claim 17, wherein the performing comprises obtaining patient feedback or patient questionnaire to assist in determining at least one of the clinical effects determinations.
